(19) United States
(12) Patent Application Publication
(10) Pub. No.: US 2003/0108925 A1
(43) Pub. Date: Jun. 12, 2003

(54) Genetic testing for male factor infertility

(75) Inventors: David Jacob Dix, Raleigh, NC (US); Stephen A. Krawetz, Detroit, MI (US); David Miller, Leeds General Infirmary (GB)

Correspondence Address: BROWDY AND NEIMARK, P.L.L.C., 624 Ninth Street, N.W., Washington, DC, 20001, US

(73) Assignees: U.S. EPA, Research Triangle Park, NC; Wayne State University, Detroit, MI; University of Leeds, Leeds General Infirmary (GB)

(21) Appl. No.: 10/264,321

(22) Filed: Oct. 4, 2002

Related U.S. Application Data
(60) Provisional application No. 60/327,525, filed on Oct. 5, 2001.

(51) Int. Cl. C12Q 1/68

(52) U.S. Cl. 435/6

(57) ABSTRACT

Genetic testing for male infertility or damage to spermatozoa is accomplished by providing a microarray of DNA probes with a sample of spermatozoa to determine the mRNA fingerprints of the sample; and comparing the mRNA fingerprints of the sample with the mRNA fingerprints of normal fertile male spermatozoa.

### CROSS-REFERENCE TO RELATED APPLICATIONS

The present invention claims priority from provisional application Serial No. 60/327,525, filed Oct. 5, 2001, the entire contents of which are hereby incorporated by reference.

### FIELD OF THE INVENTION

The present invention relates to methods, kits, and tools for determining fertility of a male. Specifically, the present invention relates to a method for determining male fertility through genetic analysis to determine function of spermatozoa.

### BACKGROUND OF THE INVENTION

Predicting the fertility of a male is very useful in a variety of contexts. For example, the artificial insemination industry is interested in knowing the likelihood that fertilization will occur if a female is artificially inseminated with a particular male's semen. Alternatively, human fertility clinics are concerned with achieving impregnation, and evaluating the sperm count of a male is one step in this procedure. Thus, whether in the context of animal breeding, the artificial insemination industry, or human fertility clinics, determination of the fertility of the male is very important.

Ten percent of the male population have abnormally low sperm counts, and approximately one in six couples experiences difficulty in conceiving a child. Male factor infertility accounts for 40-50% of the cases in which assisted reproductive techniques are recommended. The great majority (>98%) of infertile men actually produce sperm, but, for some reason, those sperm are often unable to fertilize an egg. Chromosomal anomalies are associated with approximately one third of non-obstructive male factor infertility affecting some 2% of the infertile male population. Half of these men (15%) present with abnormal karyotypes, which the other half (12-15%) present with microdeletions in the Azoospermic Factor (AZF) region of Yq (i.e., DAZY/RBM).

Couples having difficulty starting a family must undergo an extensive battery of tests, including a testicular biopsy. However, it has not yet been possible to identify which couples will never conceive, so that these couples can forgo the lengthy, expensive, and ultimately futile infertility therapy and begin considering other options, such as sperm donors.

Testes-specific defects have only been demonstrated in men with sub-microscopic microdeletions of the Y chromosome encompassing one or more genes. It is reasonable to expect that as new testes-specific genes are discovered, more testes-restricted abnormalities will be revealed. Lesions affecting the X and Y chromosome, as well as autosomal recessive and imprinted genes, have been associated with oligozoospermia. These types of abnormalities, however, are rarely observed in clinics. The underlying causes of infertility in the remaining 98% of men with non-obstructive defects in spermatogenesis remain unknown. Accordingly, the majority of male factor infertilities are classified as idiopathic, indicating that other genetic factors should be considered.

With the exception of obvious defects such as azoospermia, globozoospermia, and immotile ciliary syndrome, the extreme heterogeneity of normal fertile human semen suggests that most idiopathic male factor infertility is not a result of monogenic disorders. Moreover, all known monogenic disorders that affect the testes affect other tissues to an equal or greater extent. Accordingly, it is reasonable to assume that the majority of idiopathic male factor infertility that has testes-restricted phenotypes is not monogenic, but oligo- or poly-genic in origin.

Two recent developments offer considerable promise towards identifying oligo- and/or poly-genic factors that influence male fertility. First, the discovery of mRNAs in ejaculate spermatozoa makes it possible to obtain transcriptional information from male germ cells using non-invasive procedures. It is expected that these mRNAs provide a window to past events of spermatogenesis, echoing tests for gene expression. Interestingly, data mining suggests that in addition to delivering the haploid male genome, spermatozoa also deliver a critical complement of mRNAs to the oocyte. Secondly, microassays make it possible to construct detailed gene expression profiles.

Most laboratory investigations of semen quality are relatively poor indicators of fertility because they are subjective and predominantly rely on physiological and morphological criteria. The consequences of using immature spermatids or other compromised germ cells in intra-cytoplasmic spermatozoa induction (CISI) procedures need careful reappraisal, considering that spermatozoal mRNA is required for the production of normal offspring.

Thus, there is a need for an effective, efficient and accurate method and/or device for determining male fertility. More specifically, there is a need for a method for determining if a male is fertile using microarrays in analyzing mRNAs of spermatozoa.

### SUMMARY OF THE INVENTION

It is an object of the present invention to overcome the aforementioned difficulties of the prior art.

It is another object of the present invention to provide a method for detecting if a male is a normal fertile male using microarrays in analyzing the mRNA of spermatozoa.

It is a further object of the present invention to provide a kit for detecting normal fertile males using microarrays in analyzing the mRNA of spermatozoa.

It is yet another object of the present invention to provide markers for performing tests for detecting normal fertile males using microarrays in analyzing the mRNA of spermatozoa.

It is another object of the present invention to provide a test for determining if exposure to a toxin has adversely affected sperm.

The present invention provides a test, kit, and method for determining male fertility. This invention provides a genome-wide analysis to define the spermatozoal RNA fingerprint of a normal fertile male. The sperm-microarray methods outlined herein provide mechanisms for identifying infertile males.

The present invention uses microarray technology to monitor, in a sample of spermatozoa, the presence of transcripts (messenger RNAS) from over 2700 genes that the inventors have determined to be critical to normal fertility. A microarray chip is created by depositing onto slides microscopic quantities of the genetic material from these genes, and then overlaying onto the slides the genetic material extracted from a sample ejaculate. If complementary genetic material is present in the sample, it will bind to the genetic sites on the chip and be detected through laser excitation of bound fluorescence probes.

In a similar manner, the invention can be used as a toxicological/epidemiological screen to determine the presence of permanent or temporary damage to the spermatozoa of males exposed to environmental toxicants, as well as the identity of paternally derived messenger RNAs that are critical to early human development.

A suite of microarrays containing 27,016 expressed sequence tags (ESTs) was interrogated using cDNAs from a pool of nineteen testes; cDNAs from a pool of nine individual ejaculate spermatozoal mRNAs and cDNAs constructed from a single ejaculate's spermatozoal mRNAs.

The testes, pooled and single ejaculate DNAs hybridized to 7157, 3281, and 2784 ESTs, respectively. The testes population contained all of the ESTs identified by the cDNAs from the pooled and individual-ejaculate. The pooled ejaculate population contained all but 4 ESTs identified from the individual ejaculate.

Accordingly, profiling can be used to monitor past events, such as gene expression of spermatogenesis. Moreover, the data suggest that, in addition to delivering the paternal genome, spermatozoa provide the zygote with a unique suite of paternal mRNAs. Ejaculate spermatozoa can now be used as a non-invasive proxy for testes infertility investigations.

Current research supports the diagnosis of idiopathic infertility via spermatozoal mRNA fingerprints, and suggests that spermatozoal transcripts complementing those of oocytes are important for embryo development. Male gametes deliver more to the oocyte than the haploid male genome, and possess a greater role in orchestrating normal embryo development than has heretofore been recognized.

Microarrays were developed containing tiny sites that trap specific mRNA. When sperm is added, color changes at each trap site indicate whether the sperm includes each bit of mRNA. Almost immediately, one can scan the sperm to tell which mRNA, and which associated genes, are present.

The present invention can be used in various settings, including, but not limited to, hospitals, fertility clinics, artificial insemination and animal breeding facilities, and any other similar settings that can use a test for determining fertility of a male. Although the present invention is illustrated in a human model, one skilled in the art can appreciate that the invention is also applicable to and useful for animals other than humans.

In one application of the present invention, fertile spermatozoa are determined by determining the presence of mRNA. Thus, if mRNA is not present, then the spermatozoa are deemed to be non-functional. Additionally, the assay of the present invention is useful in toxicological screening and risk assessment to determine if a male species has suffered permanent or temporary damage to spermatozoa populations.

Additionally, the assay of the present invention can be used to identify specific mRNAs that are paternally derived and are critical to early human development. These parentally derived mRNAs include, but are not limited to, the following human uni-Gene: Hs.27695; Hs.19500; Hs.8867; Hs.46925; Hs.2714; Hs.152213; Hs.18195; Hs.274402; Hs.250899; Hs.2128; Hs.75106; Hs.86368; Hs.97633; and any other similar mRNA sequences known to those of skill in the art. Since these paternally derived mRNAs are essential to development, they serve as excellent markers.

### BRIEF DESCRIPTION OF THE DRAWINGS

FIG. 1 is a fingerprint of human testes and sperm RNAS.

FIG. 2 shows a distribution of testes and spermatozoal RNAs.

FIG. 3 illustrates spermatozoal RNA ontogeny.

FIGS. 4A-E show isolation of spermatozoal RNA.

FIG. 4F illustrates fidelity of spermatozoal RNA preparations.

FIG. 5 shows genetic profiling of ejaculate spermatozoa.

### DETAILED DESCRIPTION OF THE INVENTION

The present invention provides a window into the male reproductive system so that it is possible to monitor overall reproductive health with precision. The assays of the present invention can be used not only for predicting whether an individual is fertile or not, but also to obtain a detailed description of the gene-environment interaction for that individual. In the latter case, microarrays target genetic differences between the normal male model and men who have been exposed to suspected toxins. The microarray test provides a quick determination of whether a man's sperm had been adversely affected by a toxin. This approach takes into account not only how most people respond and the exposure limits that have been set on how most people seem to respond, but also how each individual responds. This knowledge is particularly beneficial to men who are at high risk of environmental toxin exposure through the workplace. In one example, a company could use the microarray test to monitor its male employees for overall exposure-induced changes in fertility. In another instance, an employee who was trying to start a family might request a test to ensure he was fertile. In the latter case, if a problem did arise, the employee could curtail his exposure and simply wait the average 60-90 days for his body to replace the old sperm with new, unexposed sperm.

In animal husbandry, particularly with beef production, cattle have a long gestation time. If one could ensure that every pregnancy outcome would be successful, beef production could be increased, thus increasing profit margins.

The use of microarray technology allows for the study of complex interplay of genes and other genetic material simultaneously. As is known, the pattern of genes expressed in a cell is characteristic of its state. Additionally, virtually all differences in cell states correlate with changes in mRNA levels of genes. Generally, microarray technology involves obtaining complementary genetic material to genetic material of interest and laying out the complementary genetic material in microscopic quantities on solid surfaces at defined positions. Genetic material from samples is then eluted over the surface, and complementary genetic material binds thereto. The presence of bound genetic material is detected by fluorescence following laser excitation. However, other detection means can also be used.

As known to those skilled in the art, spermatogenesis is a multifaceted developmental program beginning with mitotic divisions of diploid spermatogonia. These divisions give rise to spermatocytes, which undergo meiosis to produce haploid round spermatids. The final stage of spermatogenesis, termed spermiogenesis, is highlighted by the differentiation of round spermatides into spermatozoa. Once spermatogenesis is complete, spermatozoa are released from their chaperones, the Sertli cells, through a process known as spermiation. Throughout the elaborate process of spermatogenesis, many testes-specific mRNAs are synthesized and placed under stringent translational control to ensure appropriate temporal and spatial expression. The mRNAs observed in mature spermatozoa are remnants of untranslated stores that provide a historic record or fingerprint of spermatogenesis. Hence, the spermatazoal mRNA fingerprint representing the normal fertile male serves as a standard for identifying the causes of idiopathic infertility. Despite the obvious wealth of information contained within these repositories, the complexity and function of spermatozoal mRNAs have not been characterized. Once defined, however, this fingerprint provides information on the underlying causes of male factor infertility and the reasons mRNAs remain in mature spermatozoa, while rRNAs are most likely lost.

Several studies support the conclusion that spermatozoa contain a complex repertoire of mRNAs. Even though these mRNAs are thought to provide a window to past events of spermatogenesis, their complexity and function have yet to be established.

In one embodiment of the present invention, a set of 27,016 different expressed sequence tag probes (ESTs) was interrogated using cDNAs from testes and both pooled and single ejaculate spermatozoal mRNAs. The testes cDNAs hybridized to 7157 unique ESTs. This population contained all of the 3281 ESTs identified by the cDNAs of the pooled-ejaculate probe, which in turn contained 2780 ESTs identified by the cDNAs of the individual ejaculate probe. The data from testes and spermatozoa are coincident and define a spermatozoal mRNA fingerprint representative of a normal fertile male. As a result, the ejaculate spermatozoa can be used as a proxy for testes infertility investigations.

The biological complexity of the spermatozoal mRNAs was determined. Interestingly, a subset of these mRNAs was found to be associated with embryo development. This sub-population complemented that of the oocyte and was found to be unique to spermatozoa. The data suggest that, in addition to delivering the paternal genome, spermatozoa provide a greater role than had been believed in the orchestration of normal embryo development.

Spermatozoal mRNAs encapsulate the gene expression of spermatogenesis. The mRNAs observed in spermatozoa coincide with those found in the testes. Comparison of the human spermatozoal and tested mRNA fingerprints by microarray analyis was selected as the primary means to address this issue. Messenger RNAs were isolated from testes and ejaculate spermatozoa, and the corresponding cDNAs were hybridized to a series of microarrays containing 30,892 Expressed Sequence Tag probes (ESTs), of which 27,016 are unique. To define the fingerprints, a gene product is considered present if its hybridization signal is at least four-fold above background intensity.

### Materials and Methods

Human ejaculates were obtained from ten healthy volunteers of proven fertility and of normal semen quality as assessed by World Health Organization criteria. Nine of the samples were obtained from the Assisted Conception Unit at Leeds General Infirmary, Leeds, England. One sample was obtained from the normal fertile donor program at the Hutzel Hospital in Detroit, Michigan. All samples were obtained following full ethical approval and consent from each of the subjects.

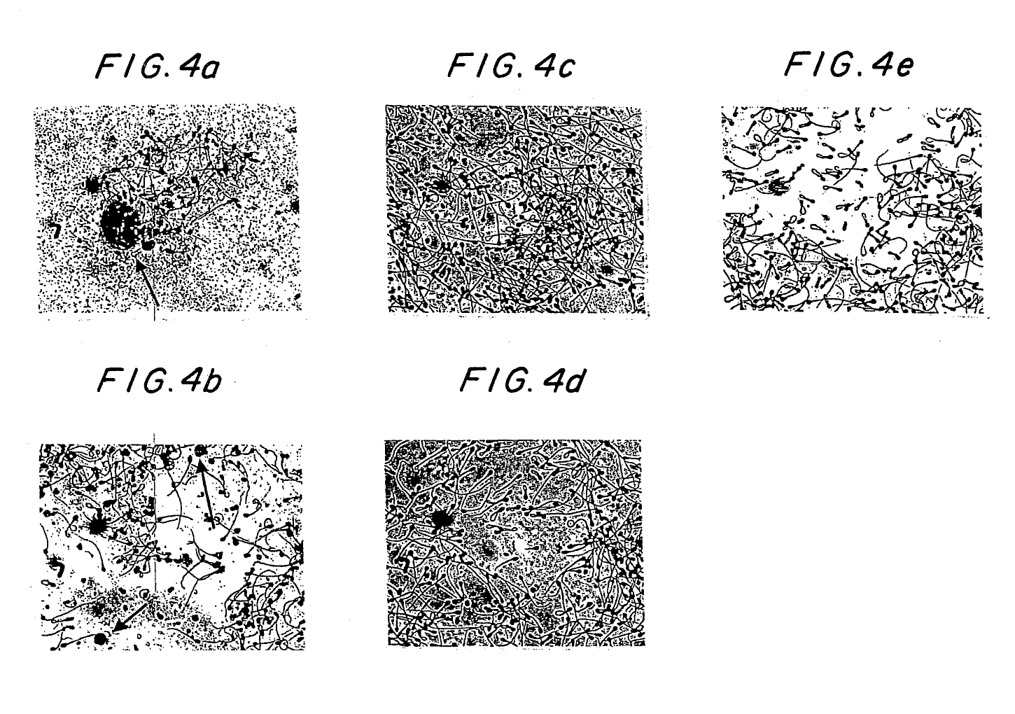

To select fertile spermatozoa and remove somatic contaminants, the nine samples from Leeds were individually purified by two sequential centrifugations through 40:80-discontinuous Percoll gradients in the following manner. Subsequent to the first centrifugation through the Percoll gradient, the pellet was resuspended, then centrifuged through a second 40:80 discontinuous gradient of Percoll. The nine spermatozoal enriched pellets were then pooled. In the unlikely event that any residual somatic contaminants were carried though, both the pooled and individual ejaculate spermatozoa were washed in a solution of 0.5% Triton X-100. The efficacy of this regimen was histochemically verified, as shown in FIGS. 4a-e. The virtual absence of ribosomal RNAs shown in FIG. 4f confirmed the lack of somatic contaminants.

In FIG. 4, a representative field of crude semen and the 40:80% Percoll interface are respectively shown in (A) and (B). Somatic cell contaminants are often and clearly observed in the crude semen (arrows). These are essentially excluded from the pellets in the first round of centrifugation (C) and are not observed in the pellets after the second round of centrifugation either before (D) or after hypotonic treatment (E).

FIG. 4F illustrates the fidelity of spermatozoal RNA preparations. Ribonucleic acid was isolated from both spermatozoa and a somatic tissue (kidney). A 5 microgram aliquot of total RNA from each preparation was loaded into separate wells of a 1.8% agarose gel. Following electrophoresis, the gel was stained with ethidium bromide. The virtual absence of 28s and 182 rRNAs in the spermatozoal preparation confirms the lack of somatic contamination.

Poly (A+) RNA was exclusively isolated from the pooled spermatozoal RNA using oligo (dT)-coated magnetic beads, as described by the manufacturer (Dynal Corp., UK). Any residual DNA was removed by treating the isolated total RNA with Rnase-free Dnase 1. The purity and integrity of both preparations of spermatozoal RNAs was verified by RT-PCR using the intron spanning protamine 2 (PRM-2) primer pair. As previously shown, the sole existence of the intronless PRM-2 amplicon verified the integrity of both the poly (A+) enriched and total RNA preparations and demonstrated that they were essentially free of DNA (Miller et al., 1999).

Complementary DNA from pooled histologically normal human testes RNAs, from 19 trauma victims ranging in age from 19 to 61 years, was purchased from Clontech Laboratories, Palo Alto, Calif. These preparations essentially contained >70% spermatogenic cells (Kramer et al., 2000). Complementary DNAs for microarray analysis were prepared from the testes and spermatozoal RNAs by reverse-transcription of 2 microgram total or poly(A+) RNA using an oligo deoxythymidine (dT) primer in the presence of 20 microliters [α-³²P]-dCTP (3000 Ci/mmol, ICN Pharmaceuticals Inc., Costa Mesa, Calif.), according to the array manufacturer's protocol (Research Genetics, Inc., Huntsville, Ala.). Labeled cDNA from 2 micrograms of total or poly(A+) RNA was evenly distributed between six arrays for hybridization.

Human Genefilter® microarrays 200, 201, 202, 203, 204 and 211 were purchased from Research Genetics, since they provided a sufficient coverage depth of the human genome and are subject to stringent quality control. This filter set contained over 30,000 sequence verified human cDNAs, each representing at least a 1 kb region of the 3′UTR (Taylor et al., 2001; Wang et al., 2000). Probes were hybridized to the filters as described by the manufacturer (http://www.resgen.com/products/GF200-protocol/php3) The filters were washed and exposed to Kodak phosphor-imaging screens for three to seven days. Images were captured using a Molecular Imager FX (Bio-Rad Laboratories, Hercules, Calif.). After control point insertion (Reid et al, 2000), the images were analyzed using Pathways software version 3.0 for Windows or UNIX (Research Genetics; Huntsville, Ala.). Hybridization of each of the three RNA samples was carried out on each of six arrays, i.e., three samples, six arrays, 18 hybridizations.

An EST was designated as present if it was at least four fold above background. This provided an efficient means to discern abundant mRNAs. The resulting binomial distribution (Conover, 1980) was then used to calculate confidence intervals and to determine the measurement error for the number of ESTs identified.

The hybridization error rate was estimated by obtaining a summation of positive hybridization signals within each of 2994 sets of ESTs that were spotted at least two times across the entire set of filters. The percent of positive hybridization signals for each set was calculated by dividing the sum of positive signals by the total number of times that the specific EST was spotted. The error rate for each set of duplicate ESTs was determined by subtracting the percent positive from 1. If an EST was spotted multiple times and all hybridization signals were negative the percent positive was set to 100, leading to an error rate of 0%.

To identify the number of unique ESTs and overlapping gene clusters in the testes and spermatozoal samples, the accession codes, gene cluster Ids and gene manes for the positive ESTs were analyzed using the Statistical Analysis Software package (SAS various 7-1; SAS Institute, Cary, N.C.). Using the sort command within SAS, duplicate accession codes within and across filters were deleted for each sample. The unique accession codes within each sample were then compared among all samples using a Boolean search strategy, and the number of shared observations was determined (Ostermeier et al., 2002, in press).

Onto-Express, a JAVA based program developed for this study (Khatri et al., 2002), was used to mine the current databases to classify the biological expression profiles of each EST. In brief, locus link was queried (ftp://ncbi.nim.nih.gov/refseq/LocusLink/LLtmpl) and the biochemical function, cellular component, and biological process of the corresponding protein was obtained.

### Results and Discussion

Throughout the multifaceted developmental program of spermatogenesis, many testis-specific mRNAs are synthesized and placed under stringent translational control to ensure appropriate temporal and spatial expression (Hecht, 1998). It has been suggested that the mRNAs observed in mature spermatozoa are remnants of untranslated sorts, and that these provide a historic record or fingerprint of spermatogenesis (Miller et al., 1994). If correct, the mRNAs observed in spermatozoa would coincide with those found in testes. Comparison of the human spermatozoal and testes mRNA fingerprints by microarray analysis was selected as the primary means to test this tenet and validate the dataset.

A wide range of mRNAs, shown in FIG. 4f, were isolated from pure preparations of spermatozoa, FIGS. 4d-e (Miller et al., 1999). The virtual absence of rRNAs (Bettach et al., 1976) in the spermatozoal preparation in comparison with the kidney control, indicates its quality. In previous studies using both differential display and gene-specific RT-PCR (Kramer et al., 1997), the presence of spermatozoal-specific RNAs in the fertile ejaculate was demonstrated by their absence in the ejaculates of vasectomized men. This and more recent data suggest that any residual rRNAs, if present, arise if pure populations of spermatozoa are processed in sufficient numbers, as was the case in the studies reported herein.

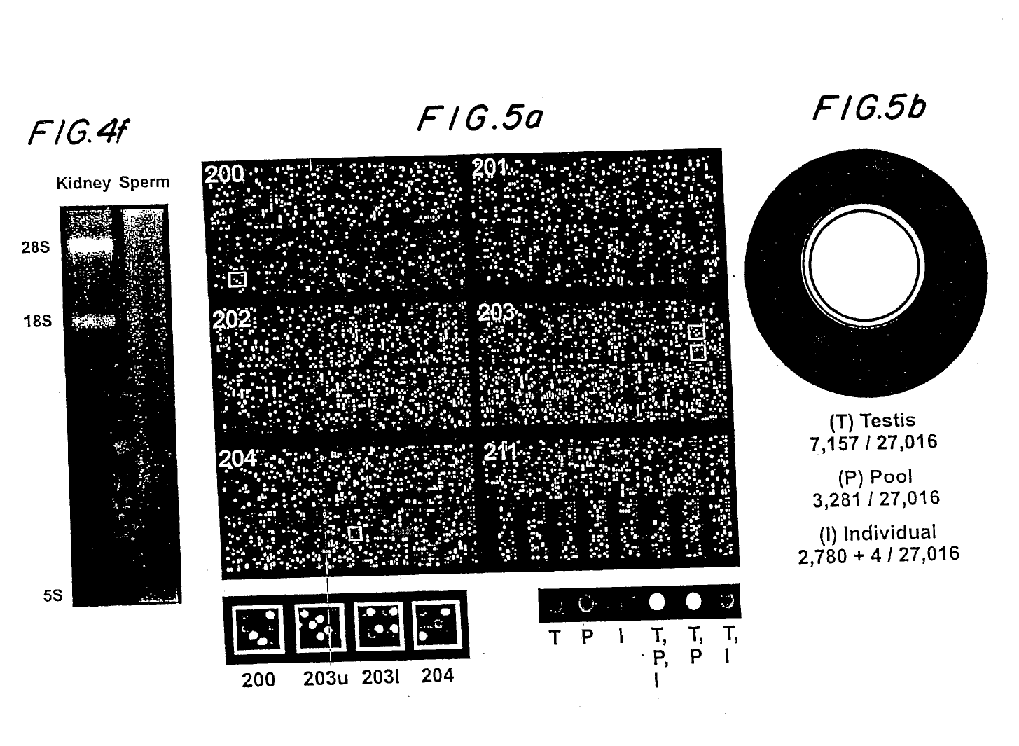

The corresponding cDNAs prepared from testes and ejaculate spermatozoa mRNAs were hybridized to a series of microarrays containing 30,892 Expressed Sequence Tag (EST) probes, of which 27,016 were unique. To define the fingerprints, an mRNA was considered present if its hybridization signal was at least four-fold above the background intensity. A summary is presented in FIG. 5a.

The hybridization error was estimated as a function of the sum of the hybridization signal present or completely absent from each of the 2994 sets of ESTs that were spotted at least twice across the entire set of filters. This analysis supported the view that the likelihood of incorrectly identifying a positive signal was 8%. To verify hybridization specificity independently, all testis associated cluster identification numbers obtained from the UniGene database (http://www.ncbi.nim.nih.gov/UniGene/) were compared to those identified on the microarray with the testes probes. Of the 9052 testis-expressed UniGene cluster identification numbers represented on the filter, a total of 3205, or 35.4%, were identified by the testis cDNA, with a hybridization signal threshold of at least four fold above background. This directly reflects the distribution of abundant mRNAs that were identified using a strict cutoff of at least four fold above background. The data are consistent with the view that the estimation of the number of transcripts constituting the testis transcription was both conservative and reliable.

To determine the extent of similarity between the testis and spermatozoal mRNAs, the unique ESTs identified using the pooled-ejaculate spermatozoa cDNA probe from nine individuals was compared to the ESTs identified using the 10 individual pooled-testes cDNA probe from a single individual. Any EST considered positive in the pooled-ejaculate and not in the testes, or identified in the individual ejaculate but not in the pooled-ejaculate and testes, were noted. The testes probe identified 7157 unique ESTs. This population fully described those identified in spermatozoa when either the pool of poly(A+) enriched RNAs or total RNA from an individual ejaculate was used as the probe. All but four of the ESTs from the 2784 identified in the individual ejaculate were contained within the 3281 ESTs identified by the pooled-ejaculate, as shown in FIG. 5B.

To ensure that this observation truly reflected spermatozoal RNAs, the purity of the spermatozoa RNA was independently assessed by comparing the spermatozoa RNAs to RNAs of lymphocyte origin (http://www.ncbi.nim.nih.gov/UniGene/;). Of the 865 lymphocyte-expressed UniGene cluster identification numbers represented by the ESTs on the microarray filters, only 5% were shared with the 2906 UniGene cluster identification numbers identified with the pooled spermatozoa probe. As expected, the gene products represented by these shared cluster identification numbers corresponded to products of ubiquitously expressed “house-keeping” genes. This indicates an essentially pure population of spermatozoal RNAs free of contamination from lymphocytes or other somatic cells. The concordance displayed between the testes and spermatozoal RNAs supports the view the spermatozoal RNAs can be used to monitor past events of gene expression during spermatogenesis.

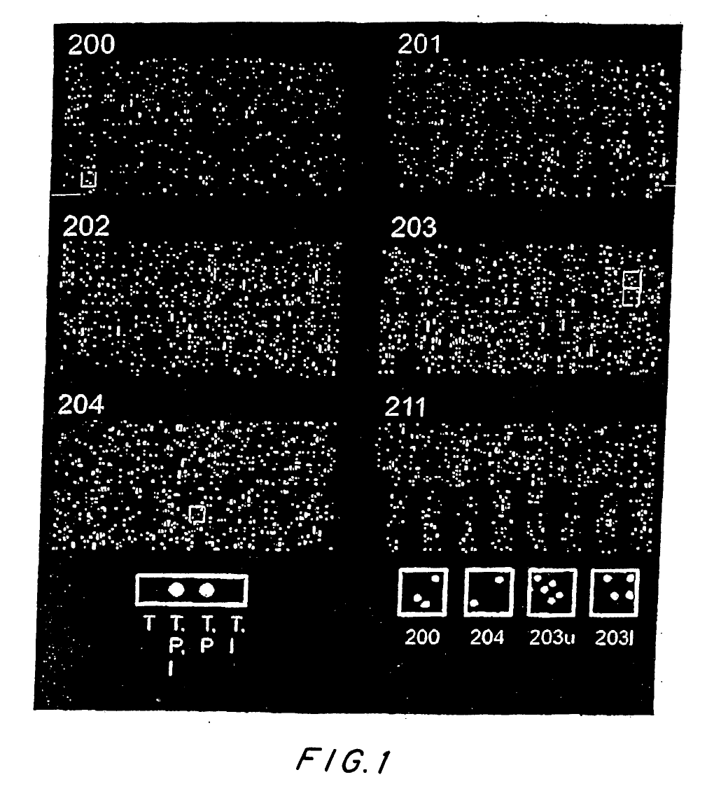

To obtain the data shown in FIG. 1, complementary DNAs, representing the mRNAs isolated from human testis and both pooled and individual ejaculate spermatozoal samples were hybridized to a series of microarrays. Each numbered panel identified the specific Gene Filter® (Research Genetics). Those expressed sequence tags (ESTs) hybridized by tested cDNAs (T) are shown in red, those hybridized by the pooled-ejaculate cDNAs (P) are shown in green, while those hybridized by the individual-ejaculate cDNAs (I) are shown in blue. When the T, P, or I filter overlap, specific colors are generated, as shown by the color-keys at the bottom of each image.

The white boxes shown in FIG. 1 show the four ESTs that hybridized to the individual but not to the pooled ejaculate cDNAs. These regions are enlarged and labeled by their corresponding Gene Filter® in the bottom right corner of FIG. 1. The upper (u) and lower (l) boxes on Gene Filter® 203 are indicated therein.

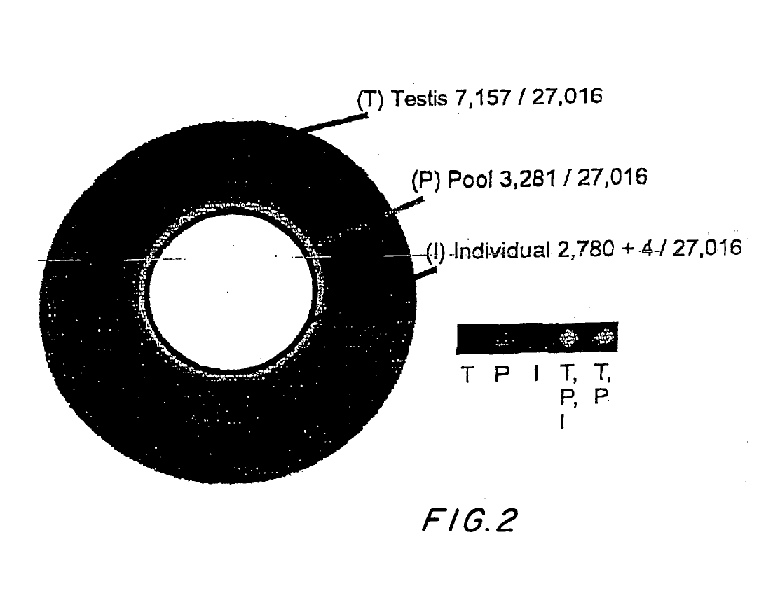

FIG. 2 shows the distribution of testes and spermatozoal RNAs. Of the 27,016 unique ESTs scanned, 7157 were identified as testes (T) cDNAs (red). The testes population contained all 3281 ESTs hybridized by the pooled ejaculate (P) cDNAs (green), which in turn contained 2780 ESTs hybridized by the individual ejaculate (I) cDNA (blue). The four ESTs identified by the individual ejaculate cDNAs but not pooled are contained within the testes population.

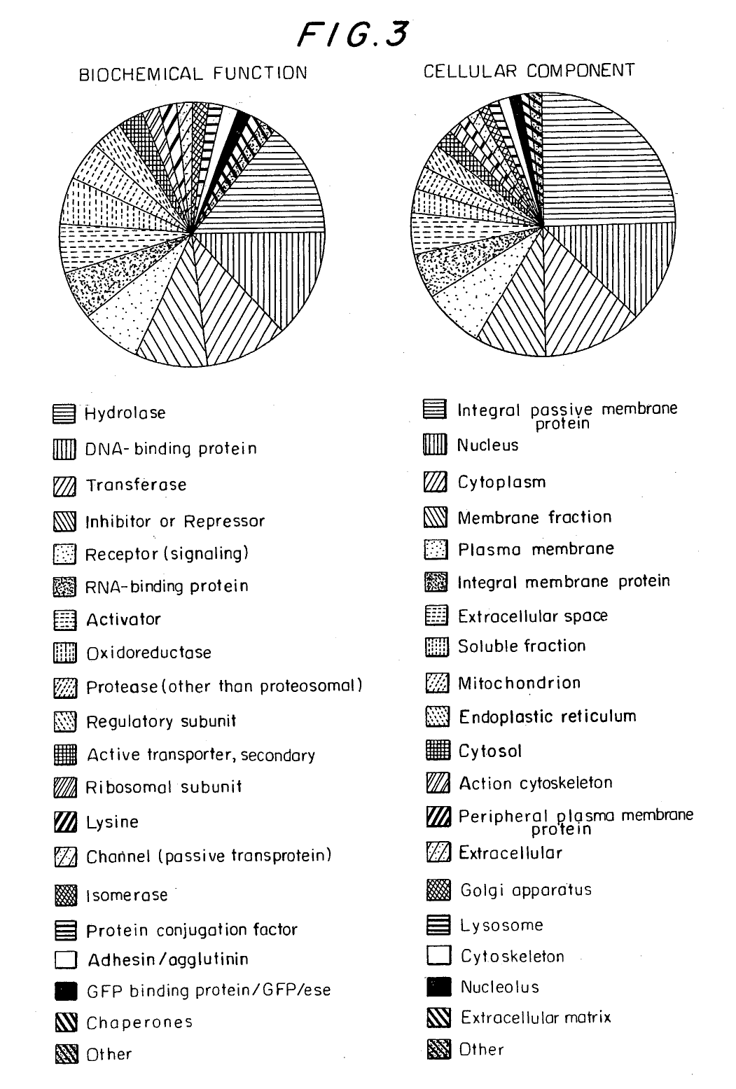

FIG. 3 illustrates spermatozoal RNA ontogeny. The biological activity of the proteins that represent each expressed sequence tag identified by the pooled-ejaculate spermatozoal cDNA was data mined using Onto-Express. The biochemical function delineates the principal structure, regulatory, or enzymatic function of the protein. The cellular component describes the location in the cell in which the protein is active. The term “other” indicates protein groups with fewer than 14 observations.

To determine the extent of similarity between the testes and spermatozoal mRNAs, the unique ESTs from a pooled-ejaculate spermatozoal cDNA probe from nine individuals, were compared to ESTs identified by a pooled-testes cDNA probe from 19 individuals, and to the ESTs identified using a spermatozoa cDNA probe from a single individual. Using the sort command within the Statistical Analysis Software (SAS version 7-1; SAS Institute, Cary, N.C.), duplicate accession codes within and across filters were deleted for each sample. The unique accession codes within a sample were compared among the samples using a Boolean search strategy, and the number of observations shared was determined. Any EST considered positive in the pooled-ejaculate and not in the testes cDNAs or identified in the individual-ejaculate but not in the pooled-ejaculate and testes cDNAs was noted. The testes cDNAs identified 7157 unique ESTs. This population fully described those identified in spermatozoa when either the pool of poly(A+) enriched RNAs or total RNA from an individual ejaculate was used as the probe. All but four of the ESTs from the 2784 identified in the individual ejaculate cDNAs were contained within the 3281 ESTs identified by the pooled-ejaculate cDNAs, as shown in FIG. 2. These data support the view that spermatozoal RNAs can be used to monitor past events, such as gene expression or spermatogenesis.

The measurement error of the spermatozoal mRNA fingerprint, at the 99% confidence level, was calculated to be within 0.80% of the ESTs identified by the pooled-ejaculate cDNA. When the population of the ESTs identified by pooled-ejaculate cDNAs was compared to those of the single ejaculate, the observed error was only four ESTs. This value is six-fold less than the calculated measurement error, indicating that a maximum number of ESTs were identified by the pooled-ejaculate cDNAs. Of the possible 27,016 unique ESTs, the individual ejaculate cDNA identified 2784 shared ESTs. Thus, it is predicted with 99% confidence that cDNAs derived from a normal fertile man's ejaculate spermatozoa hybridize to at least 2686, but to no more than 2882, of the possible 27,016 ESTs. Accordingly, a specific population and range of ESTs have been defined for this set of Gene Filter® arrays. These transcripts represent the spermatozoal fingerprint for the normal fertile male. Furthermore, these fingerprints have rapidly defined those transcripts present in spermatozoa, without constructing or sequencing the corresponding cDNA library. Thus, the present invention can be used to describe the distribution of transcripts in never before described cell populations.

Characterization of the fingerprint of the normal fertile male using OntoExpress (Khatri et al., 2002) was undertaken to address why mature spermatozoa, that are transcriptionally dormant, contain this complement of mRNAs. The biological function, cellular component, and biological process of the translated proteins corresponding to the spermatozoal mRNAs were defined for each of the hybridizing ESTs (http://compbio.med.wayne.edu/microarray). The majority of spermatozoal mRNAs participate in signal transduction, oncogenesis and cell proliferation corresponding to nuclear and plasma membrane proteins (Balhorn et al., 1999). Genes expressed early in spermatogenesis were also identified in mature spermatozoa.

In one embodiment of the present invention, characterization of the fingerprint of the normal fertile male using Onto-Express can be undertaken to shed light on the basis behind mature spermatozoa, which are transcriptionally dormant and have no rRNAs, yet contain mRNAs. Onto-Express, a JAVA based program developed for the present study, was used to mine the current databases for ontogeny and the biological expression profiles of each EST.

In brief, the locus link is queried and the biochemical function, cellular component, and biological process of the corresponding protein are obtained. The term “UNKNOWN” indicates that the biochemical function, cellular component, or biological process had not been determined. If either the cluster identification or locus link could not be obtained, the data are returned as “UNAVAILABLE.”

The biological function, cellular component, and biological process of the translated proteins corresponding to the spermatozoal mRNAs are defined for each of the hybridizing ESTs. As shown in FIG. 3, hydrolyases and DNA-binding proteins are the functional biological groups having the largest number of identified members. This is consistent with spermatozoal mRNAs encapsulating spermatogenic gene expression, as hydrolytic enzymes found in the acrosomes are translated late in spermatogenesis, and spermatid chromatin undergoes significant restructuring.

The cellular compartments represented by the largest number of identified proteins are the plasma membrane, nucleus, and cytoplasm. The concentration of cytoplasmic protein encoding mRNAs was unexpected, considering that mature spermatozoa have little cytoplasm. This can be reconciled in the following manner. First, proteins localizing to the cytoplasm may function in the developing germ cell wall before the cytoplasmic reduction at spermiation. Several genes expressed early in spermatogenesis have been identified in mature spermatozoa. Testis specific protein Y-linked, an early expressed gene, and testis IN, a gene expressed prior to meiosis, are identified in the testes and both the pooled-ejaculate and single-ejaculate probes.

Examples of additional mRNAs expressed relatively early in spermatogenesis and identified both in the testes and spermatozoal cDNA probes include: tubulin, al (testes specific); amiloride-sensitive cation channel 3, testis; t-complex-associated-testis expressed 1-like; t-complex associated testis expressed 1-like 1; testis specific protein 1 (probe h4-p3-1); phosphodiesterase 1B (previously identified in sperm). This suggests that numerous spermatozoal mRNAs are assembled and maintained throughout spermatogenesis. Alternatively, these stores of spermatozoal mRNAs may provide function in a manner similar to that established in oocytes and may be necessary for sustaining zygotic and/or embryonic viability prior to the activation of the embryonic genome.

As shown in Table 1, a series of spermatozoal mRNAs is identified that participate in fertilization and embryonic development. These proteins include a group associated with fertilization; several heat shock response products, which are important for embryo development; a series that function in embryogenesis and morphogenesis as well as implantation. This was found to be rather intriguing, considering that spermatozoa were believed to contribute little more than the paternal genome, a calcium bob for activating oocytes, and centrioles.

TABLE 1
Spermatozoal mRNA and their function in early development
Biological Process

| | Heat Shock | Embryogenesis and | |
|---|---|---|---|
| Fertilization | Response | Morphogenesis | Implantation |
| CLU^{a} | HSF2^{e} | MID1¹ | RPL2^{r} |
| CLGN^{b} | HSPA1B^{f} | NLVCF^{k} | |
| AKAP4^{c} | DNAJB1^{g} | CYR61¹ | |
| GNP1^{d} | HSBP1^{h} | EYA3^{m} | |
| | DUSP5¹ | FOXG1Bⁿ | |
| | | WNT5A^{o} | |
| | | WHSC1^{p} | |
| | | SOX13^{q} | |

¹The biological “objective” to which the protein contributes;
^{a}Clusterin (complement lysis inhibitor, SP-40,40 sulfated glycoprotein 2, testosterone-repressed prostate message 2, apolipoprotein j)
^{b}Calmegin;
^{c}A kinase (PRKA) anchor protein 4;
^{d}Glucosamine-6-phosphate isomerase;
^{e}Heat shock transcription factor 2;
^{f}Heat shock 70 kD protein 1B;
^{g}DnaJ (Hsp40) homolog, subfamily B, member 1;
^{h}Heat shock factor binding protein 1;
ⁱDual specificity phosphatase 5;
^{j}Midline 1;
^{k}Nuclear localization signal deleted in velocardiofacial syndrome;
^{l}Cysteine-rich, angiogenic inducer, 61;
^{m}Eyes absent (Drosophila) homolog;
ⁿForkhead box G1B;
^{o}Wingless-type MMTV integration site family, member 5a;
^{p}Wolf-Hirschhorn syndrome candiate 1;
^{q}SRY (sex determining region Y)box 13);
^{r}Ribosomal protein L29

Even though mammalian male and female pronuclei can be considered genetically equivalent, studies indicate that they are indeed functionally different. Human females can present with a hydatidiform mole, in which the fetus is absent and the placental tissue is abnormally enlarged. A majority of these moles arise when a haploid spermatozoa fertilizes and oocyte lacking the maternal pronucleus. Furthermore, when mouse androgenones and gynegenones are produced, the embryos do not develop far beyond the blastocyst stage and fail from their respective deficiencies in chorion and embryo proper. This supports the view that the spermatozoa and oocyte contribute distinct functionalities to the developing embryo that go beyond imprinting.

To test whether or nor spermatozoal mRNAs are required for zygotic and/or embryonic development, the spermatozoal mRNAs were compared to the population of mRNAs previously identified in oocytes. It is reasoned that if spermatozoa mRNAs are queried, they would be absent in oocytes. When the Unigene cluster identification numbers (representing spermatozoal mRNAs) were compared to cluster identification numbers from oocyte mRNAs, no duplicate values were identified. This indicates that spermatozoa provide novel transcripts distinct from those of the oocyte consistent with the view that they are essential for zygotic and/or embryonic development.

Furthermore, when the UniGene database was searched for mouse homologues corresponding to these human transcripts, no evidence was found to indicate that these populate the female gamete. This suggests that spermatozoa provide novel transcripts distinct from those of the oocyte. Indeed, when polymerase chain reactions were carried out using cDNA pools obtained from zygotes that failed in vitro fertilization, all of the in silico identified transcripts but A kinase (PRKA) anchor protein 4 were present. Thus, in addition to encapsulating spermatogenic gene expression, spermatozoa mRNAs may provide a function similar to that established for the population of stored oocyte mRNAs (Latham, 1999). They may be necessary for sustaining zygotic and/or embryonic viability prior to or subsequent to the activation of the embryonic genome. This function is consistent with the major biological processes identified for the spermatozoal RNAs. Accordingly, this store of mRNAs may enable men to play a greater role in human development than has previously been considered.

Now that the spermatozoal RNA fingerprint of the normal human fertile male has been identified, it is now possible to identify and diagnose idiopathic infertilities using spermatozoal mRNA fingerprints. The normal fertile male spermatozoal fingerprint can serve as a standard to inform on the underlying causes of male factor infertility.

The foregoing description of the specific embodiments will so fully reveal the general nature of the invention that others can, by applying current knowledge, readily modify and/or adapt for various applications such specific embodiments without departing from the generic concept, and, therefore, such adaptions and modifications should and are intended to be comprehended within the meaning and range of equivalents of the disclosed embodiments. It is to be understood that the phraseology or terminology employed herein is for the purpose of description and not of limitation.

### REFERENCES

Bhasin S; DeKrester D M, Baker H W G, Clinical Review 64: Pathophysiology and Natural History of Male Fertility, J. Clin, Endocrinol Metab. 1994 79:1525-1529

Van Assche E, Bonduelle M, Tournaye H, Joris H, Verheyen G, Devroey P, Van Stierteghem A, Liebaers I Cytogenitics of infertile Men Hum. Reprod. 1996; Suppl 4:1-26

Kuroda-Kawaguchi T, Skaletsky H, Brown L G, Minx P J, Cordumn H S, Waterston R H, Wilson R K, Silber S, Oates, Rozen S, Page D C, The AZFcregion of the Y Chromosome Features Massive Palindromes and Uniform Recurrent Deletions in Infertile Men Nat. Genet 29: 279-286, 2001

Lissens W Genetics of Male Reproductive Dysfunction in Molecular biology in Reproductive Medicine, Ed. Fauser, B C J M, 1999; pp. 4790594

Kramer J., Krawetz S A RNA in Sperm: Implications within the Sperm Genome, Miol. Hum. Reprod. 1997; 3:473-478

Miller D, Tang P Z, Skinner C, Lilford R J, Differential RNA Fingerprinting as a Tool in the Analysis of Spermatozoal Gene Expression Hum Reprod. 1995 9:864-869

Miller D, Briigs D, Snowden H, Hamlington J, Follison S, Lilford R, Krawetz S A. A Complex Population of RNAs in Human Ejaculate Spermatozoa: In placations for Understanding Molecular Aspects of Spermiogenesis Gene 1999 237:385-392

Richter W, Dettmer D, Glander H J Detection of mRNA Transcripts of Cyclic Nucleotide Phosphodiesterase Subtypes in Ejaculated Spermatozoa Mol Hum. Reprod. 1999 5: 732-736

Iver V R, Eisen M B, Ross D T, Schuler G, Moore T, Lee J C, Trent J M, Staudt L M, Hudson H Jr, Boguski M S, Lashari D, Shalon D, Botstein D, Brown P O, The Transcriptional Program in the Response of human Fibroblasts to Serum, Science 1999 283:83-87

Bittner M, Meltzer P., Chen Y, Jiang Y, Seftor E, Hendrix M, Radmacher M, Simon R, Yakhini Z, Ben-Dor A, Sampas N, Dougherty E Wang E, Marincola F, Gooden C, Lueders J, Glargelter A, Pollock P. Carpten J, Gilanders E, Leja D, Dietrich K, Beaudry C, Berens M, Alberts D, Sondak V, Molecular Classification of Cutaneous Malignant Melanoma by Gene Expression Profiling Nature 2000 406: 536-540

Beitlach C J, Erickson R P 28 S and 18 S Ribonucleic Acid from Mammalian spermatozoa J Exp Zool 1976, 198:49-55

Kramer J A, McCarrey J R, Djakiew D, Krawetz S A. Human Spermatogenesis as a Model to Examine Gene Potentiation Molecular Reproduction and Development 2000 56: 254-258

Taylor E, Cogdell D, Coombes K, Hu L, Ramdas L, Tabor A, Hamilton S, Zhang W Sequence Verification as Quality-control step for Production of cDNA Microarrays Biotechniques 2001 31:62-65

Wang E, Miller L D, Ohnmacht G A, Liu E T, Marincola F M, High-Fidelity mRNA Amplification for Gene Profiling Nature Biotech. 2000 18:457-459

Reid R, Dix D J, Miller D, Krawetz S A Recovering Filter-based Microarray Data for Pathways Analysis Using a Multipoint Alignment Strategy Biotechniques 2001 30:2-6

Conover W J Confidence Interval for a Probability in: Practical Nonparametric Statistics, 2nd Edition John Wiley & Sons, 1980 pp.99-105

Ostermeier G C, Dix D J, Krawetz S A. A Bioinformatic Strategy to Rapidly Characterize cDNA Libraries Bioinformatics 2002 (in press)

Khatri P, Draghici S, Ostermeier G C, Krawetz S A, Profiling Gene Expression Utilizing Onto-Express Genomics 2002 79:166-270

Hecht N. Molecular Mechanisms of Male Germ Cell Differentiation Bioassays 1998 20: 555-561

Ostermeier, G C. Sperm Nuclear Morphology and its Relationship with bull Fertility. Madison, Wis.: University of Wisconsin; Ph.D. Dissertation 2000.

Balhorn, R. et al. Protamine mediated condensation of DNA in mammalian sperm. In: The Male Gamete: From Basic Science to Clinical Applications. (Edt. By Gagnon C.) cache River Press. 1999; pp. 55-70.

Dix, D. J., et al. Inhibition of hsp70-1 and hsp70-3 expression disrupts preimplantation embryogenesis and heightens embryo sensitivity to arsenic. Mol Reprod Dev. 1998;51:373-80.

Swann, K., et al. Mechanism of Ca2+ release at fertilization in mammals. J Exp Zool. 1999;285:267-75.

Sathananthan, A. H., et al. The sperm centriole: its inheritance, replication and perpetuation in early human embryos. Hum Reprod. 1996;11:345-56.

Simerly, C., et al. The paternal inheritance of the centrosome, the cell's microtubule-organizing center, in humans, and the implications for infertility. Nat Med. 1995;1(1):47-52.

Jacobs, P. A., et al. Mechanism of origin of complete hydatidifom moles. Nature. 1980;286:714-716.

McGrath, J., et al. Completion of mouse embryogenesis requires both maternal and paternal genomes. Cell. 1984;37:179-183.

Neilson, L., et al. Molecular phenotype of the human oocyte by PCR-SAGE. Genomics. 2000;63:13-24.

Stanton, J. L., et al. A set of 840 mouse oocyte gene with well-matched human homologues. Mol Human Repro. 2001;7:521-543.

Latham, K. E., Mechanisms and control of embryonic genome activation in mammalian embryos. Int Rev Cytol. 1999;193:71-124.

What is claimed is:

1. A method for testing for male factor infertility comprising:
contacting a microarray of DNA probes with a sample of spermatozoa to determine the mRNA fingerprints of the sample; and
comparing the mRNA fingerprints of the sample with the mRNA fingerprints of normal fertile male spermatozoa.

2. The method according to claim 1 wherein the male is a human male.

3. The method according to claim 1 wherein the expressed sequence tags are identified fluorometrically.

4. The method according to claim 1 wherein a DNA probe is considered to be addressed if its hybridization signal is at least four-fold above background intensity.

5. A method for testing for exposure to toxins that interfere with male reproduction comprising:
obtaining a sample of spermatozoa;
contacting a microarray of expressed sequence tag probes with the sample to determine the mRNA fingerprints of the sample; and
comparing the mRNA fingerprints of the sample with mRNA fingerprints of normal fertile male spermatozoa to determine which, if any, mRNA has been damaged by exposure to toxins.

6. A method for identifying mRNAs that are paternally derived comprising applying a sample of ejaculate spermatozoa to a microarray of mRNAs that are paternally derived and detecting which mRNAs are addressed.

7. The method according to claim 6 wherein the mRNAS are selected from the group consisting of Hs.27695; Hs.19500; Hs.8867; Hs.46925; Hs.2714; Hs.152213; Hs.18195; Hs.274402. Hs.250899; Hs.2128; Hs.75106; Hs.86368; Hs.97633; and combinations thereof.

8. A kit for assay of spermatozoa comprising a DNA microarray comprising the identified mRNAs of normal fertile sperm.
